(19) United States
(12) Patent Application Publication
(10) Pub. No.: US 2025/0209275 A1
(43) Pub. Date: Jun. 26, 2025

(54) SYSTEMS, METHODS, AND ARTICLES FOR ENHANCING THE TRAINING OF NATURAL LANGUAGE PROCESSING MODELS IN BIOMEDICAL CONTEXT

(71) Applicant: Tempus AI, Inc., Chicago, IL (US)

(72) Inventor: Tian Kang, New York, NY (US)

(21) Appl. No.: 18/393,312

(22) Filed: Dec. 21, 2023

(51) Int. Cl. G06F 40/30; G16H 10/60

(52) U.S. Cl. CPC G06F 40/30; G16H 10/60

(57) ABSTRACT

Technologies for enhancing natural language processing (NLP) model training in biomedical context are disclosed. An example method includes training an embedding model to capture semantic richness of biomedical context based on unstructured texts obtained from an electronic health record (EHR) system, obtaining a seed set of seed texts and an unlabeled set of unlabeled texts, using the trained embedding model to determine a vectorized semantic representation for each seed text and each unlabeled text, assigning classification labels to at least a subset of the unlabeled set by clustering the seed set with the unlabeled set based on the vectorized semantic representations for each seed text and unlabeled text, and providing NLP model training data including the assigned classification labels.

### BACKGROUND

### Technical Field

The present disclosure generally relates to natural language processing (NLP) modeling and training, and more particularly, to NLP training enhancement in biomedical context.

### Description of the Related Art

Advanced machine learning methods such as Deep Learning, particularly large language models (e.g., Bidirectional Encoder Representations from Transformers (BERT), Generative Pre-trained Transformers (GPT), or the like), have been successful in advancing NLP tasks. However, developing effective NLP models in biomedical context (e.g., for the development of medicine or other treatment) faces challenges. As an example, due to privacy concerns and expensive expertise in the clinical domain, it is impossible or impractical to collect labeled data of a sufficient size for model training to achieve optimal or near-optimal performance. Accordingly, the progress of biomedical NLP typically lags general domain NLP.

An electronic health record (EHR) contains information relating to patient care, often from a variety of sources. The information may include structured data, which is typically organized into fields in a database or other organizational schema. Examples of structured data include demographic information about a subject such as the subject's address, age, weight, and height. Diagnostic codes, billing codes, and laboratory test results often appear as structured data in electronic health records.

In many cases, an EHR contains unstructured data. In contrast with structured data, unstructured data (e.g., free text) does not have a predefined data model, and may include information in many different formats, many of which cannot be processed or analyzed using conventional methods. Medical or clinical notes are one example of unstructured data. Medical or clinical notes document a patient's interaction with a healthcare worker such as a physician, nurse, physician's assistant, technician, radiologist, or the like, and may be stored in an EHR. Medical or clinical notes may include consultation notes, referral notes, Subject, Objective, Assessment, and Plan (SOAP) notes, procedure notes, phone notes, or the like. Medical or clinical notes are often handwritten and may use non-standard formatting. Data such as x-ray images, mammograms, and digital pathology files often appear as unstructured data in an EHR.

While unstructured data may include detailed information about a patient encounter, factors including inconsistent formatting, quality, and data types may make unstructured data difficult to interpret and extract information from systematically. The lack of significant progress in biomedical NLP exacerbates this problem.

### BRIEF SUMMARY

Labels for data to train or otherwise develop NLP models are typically generated manually. This conventional way to accumulate labeled data is expensive and time consuming, which usually results in a limited size of training data and makes it challenging to train NLP models with the optimal performance.

Systems, methods, and articles for enhancing NLP model training in biomedical context are provided. The technologies disclosed herein include weak labeling functions with no or minimum manual efforts to collect large-scale training labels for biomedical NLP models. In some embodiments, a representative method includes: training an embedding model to capture semantic richness of biomedical context based, at least in part, on a set of unstructured texts obtained from an EHR system; obtaining a seed set including seed texts each associated with a classification label; obtaining an unlabeled set including unlabeled texts; using the trained embedding model to determine a vectorized semantic representation for each seed text of the seed set and for each unlabeled text of the unlabeled set; assigning classification labels to at least a subset of the unlabeled set by clustering the seed set with the unlabeled set based, at least in part, on the vectorized semantic representations for each seed text and for each unlabeled text; and providing NLP model training data including the assigned classification labels.

In some embodiments, the set of unstructured texts includes context snippets identified from clinical notes. In some embodiments, the context snippets are identified based, at least in part, on at least one of demographics, medical history, diagnosis, severity of disease, medication, therapy, surgery, or associated outcome.

In some embodiments, training the embedding model comprises using at least the set of unstructured texts to finetune a large language model (LLM), wherein the LLM was pretrained for general-purpose language understanding. In some embodiments, the method further includes, for each unstructured text of the set of unstructured texts: extracting one or more entities of interest from the unstructured text; and replacing at least a subset of the one or more extracted entities of interest with one or more types of mask tokens in the unstructured text to generate respective masked text. In some embodiments, the at least a subset of the one or more extracted entities is selected based, at least in part, on a task of an NLP model to be trained with the NLP model training data. In some embodiments, the task of the NLP model includes at least one of predicting diagnoses, identifying biomarker, recommending treatment, or determining medication intake.

In some embodiments, the method further includes labeling each masked text with at least one target label based, at least in part, on the one or more entities of interest extracted from the unstructured text. In some embodiments, the labeling includes normalizing the one or more entities of interest based, at least on part, on medical or clinical ontology. In some embodiments, the method further includes adapting the LLM to use each masked text to predict its associated target label. In some embodiments, the parameters of the LLM are adjusted during the adapting and fixed after the adapting is completed.

In some embodiments, given an input to the trained embedding model, a vectorized semantical representation of the input is generated based, at least in part, on output of one or more layers of the trained embedding model. In some embodiments, the vectorized semantical representation of the input is generated by averaging the output of the one or more layers.

In some embodiments, assigning classification labels to at least a subset of the unlabeled set by clustering the seed set with the unlabeled set comprises iteratively performing the clustering while expanding the seed set.

In some embodiments, the classification labels assigned to the at least a subset of the unlabeled set is based, at least in part, on one or more nearest neighbors in a finalized seed set.

In some embodiments, at least a subset of the assigned classification labels is used to generate, modify, or supplement structured texts in the EHR system.

In some embodiments, at least a subset of the assigned classification labels is used in conjunction with data obtained from EHR system as input into at least one of a classification, prediction, or association model to produce output.

In some embodiments, the NLP model training data is used to train at least one of a support vector machine (SVM), neural network, or large language model (LLM).

In some embodiments, a representative computing system includes one or more processors and one or more non-transitory computer-readable media collectively storing instructions that, when collectively executed by the one or more processors, cause the computing system to perform actions. The actions include: training an embedding model to capture semantical richness of biomedical context based, at least in part, on a set of unstructured texts obtained from an EHR system; obtaining a seed set including seed texts each associated with a classification label; obtaining an unlabeled set including unlabeled texts; using the trained embedding model to determine a vectorized semantical representation for each seed text of the seed set and for each unlabeled text of the unlabeled set; and assigning classification labels to at least a subset of the unlabeled set by clustering the seed set with the unlabeled set based, at least in part, on the vectorized semantical representations for each seed text and for each unlabeled text.

In some embodiments, a non-transitory processor-readable storage medium stores computer instructions that, when executed by one or more processors, cause the one or more processors to perform actions. The actions include: training an embedding model to capture semantical richness of biomedical context based, at least in part, on a set of unstructured texts obtained from an EHR system; obtaining a seed set including seed texts each associated with a classification label; obtaining an unlabeled set including unlabeled texts; using the trained embedding model to determine a vectorized semantical representation for each seed text of the seed set and for each unlabeled text of the unlabeled set; and assigning classification labels to at least a subset of the unlabeled set by clustering the seed set with the unlabeled set based, at least in part, on the vectorized semantical representations for each seed text and for each unlabeled text.

NLP model training data including the assigned classification labels can be used to train models with various NLP classification tasks or other tasks. For example, given a context snippet (e.g., clinical note snippet) that contains a type of entity of interest (e.g., a cancer type mention), predict whether the entity of interest is the subject patient's diagnosis or not; given a context snippet that contains a type of entity of interest (e.g., a biomarker or a gene name mention), predict whether the entity of interest's test result for the subject is positive or not; or given a context snippet that contains a type of entity of interest (e.g., a medication mention), predict whether the subject patient is taking the medication or not.

### BRIEF DESCRIPTION OF THE SEVERAL VIEWS OF THE DRAWINGS

Like-numbered elements may refer to common components in the different figures.

FIG. 1a is a flow diagram illustrating a process for enhancing NLP model training according to some embodiments of the present disclosure.

FIG. 1b shows an example tabular format of NLP model training data according to some embodiments of the present disclosure.

FIG. 2a is a flow diagram illustrating a process for training and using an embedding model to capture semantic richness of biomedical context according to some embodiments of the present disclosure.

FIG. 2b shows two examples of masked texts and their assigned labels according to some embodiments of the present disclosure.

FIG. 2c shows an example of an embedding representation generated for an input text according to some embodiments of the present disclosure.

FIG. 3 is a flow diagram illustrating a process for assigning classification labels to unlabeled texts according to some embodiments of the present disclosure.

FIG. 4 is a block diagram illustrating a computing system or device used to implement some or all the functionalities of the technology disclosed herein.

### DETAILED DESCRIPTION

Advanced machine learning methods, particularly large language models (LLMs), have been successful in advancing NLP tasks. However, developing effective NLP models in biomedical context faces challenges. As an example, labels for data to train or otherwise develop NLP models are typically generated manually. This conventional way to accumulate labeled data is expensive and time consuming. In biomedical context, this issue is more severe due to privacy concerns and expensive expertise in the clinical domain. Therefore, it is impossible or impractical to collect labeled data of a sufficient size for model training (especially modern deep learning methods which achieve state-of-the-art performance in clinical NLP tasks but require vast amount of high-quality labels) to achieve optimal or near-optimal performance. The technologies disclosed herein include weak labeling functions with no or minimum manual efforts to collect large-scale training labels for biomedical NLP models.

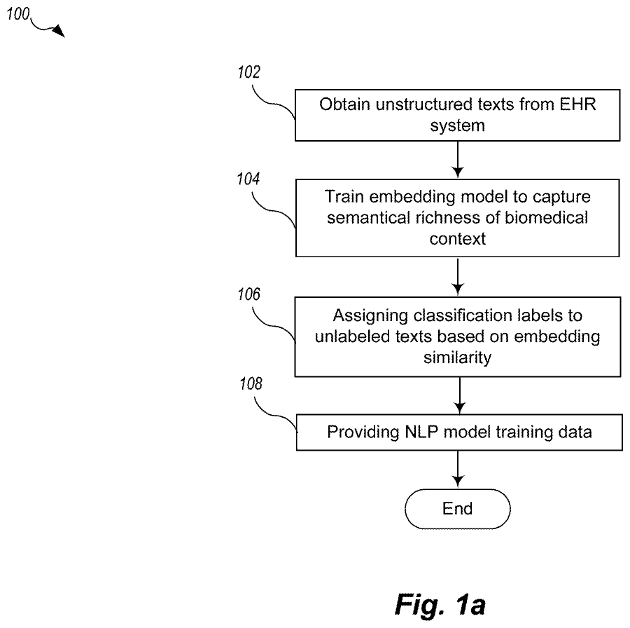

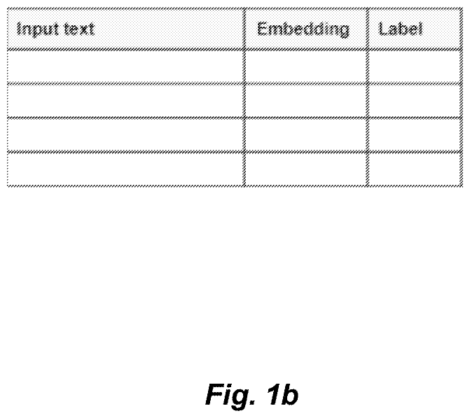

FIG. 1a is a flow diagram illustrating a process 100 for enhancing NLP model training according to some embodiments of the present disclosure. The process 100 may be performed using one or more real or virtual machines and/or one or more containerized applications, inside or outside of an EHR system. The process 100 starts at block 102, where unstructured texts are obtained from the EHR system. As described above, unstructured texts do not have a predefined data model, and may include information in many different formats, many of which cannot be processed or analyzed using conventional methods.

The unstructured texts can include context snippets identified from medical or clinical notes that are obtained from the EHR system. A context snippet, in the context of NLP, typically refers to a small segment of text that contains relevant information surrounding specific word(s), phrase(s), or other entity (or entities) of interest within a larger body of text or dataset. The snippet is often extracted to provide context or additional information about the entity (or entities) being analyzed. The medical or clinical notes can be broken down into such digestible pieces by identifying which snippets of text relate to entities of interest (e.g., desired terms) for extraction and use. Such terms can include demographics of the subject, medical history of the subject, medical history of the subject's family, diagnosis of disease states, severity of those disease states, medications, therapies, surgeries, associated outcomes, combination of the same or the like.

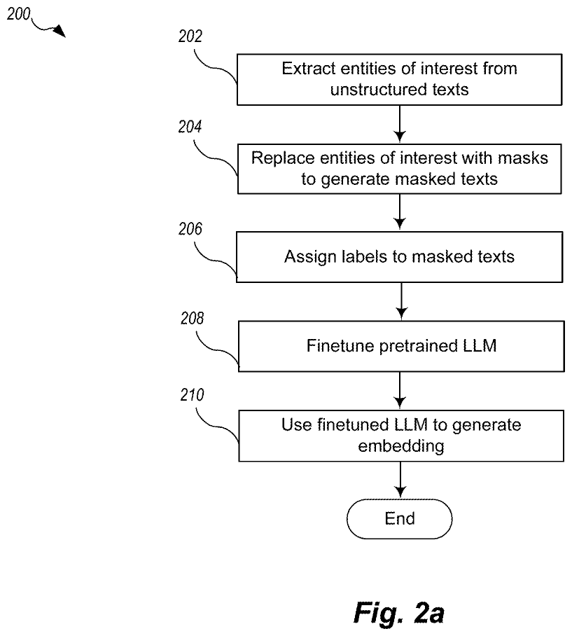

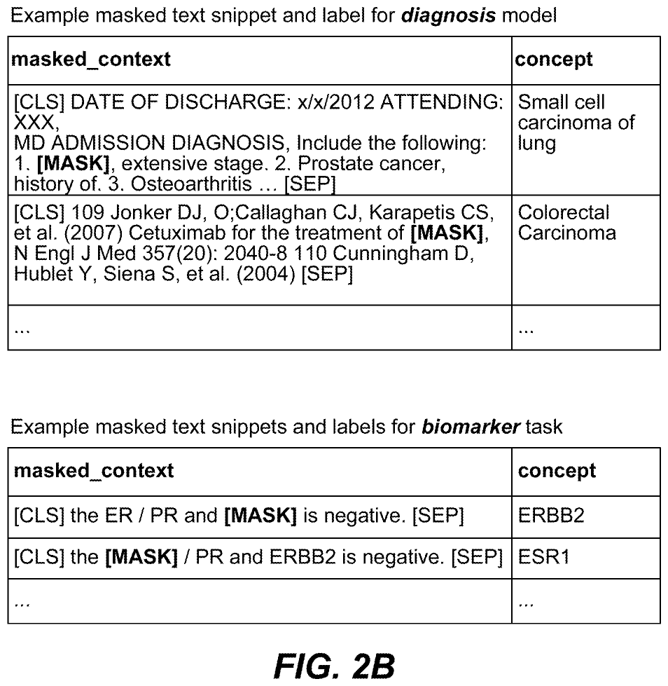

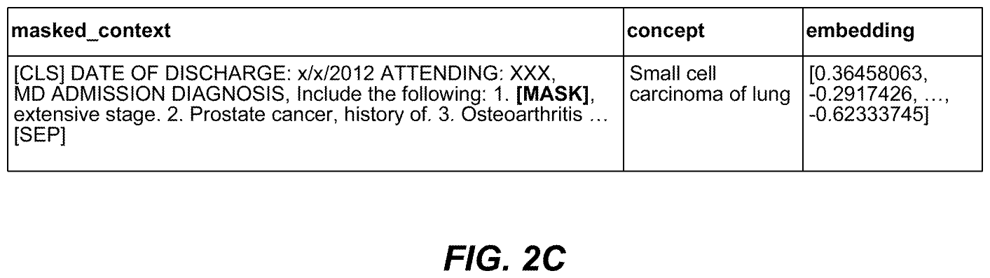

As will be described in more detail with reference to FIG. 2a, entities of interest can be identified and extracted from individual unstructured texts, and correspondingly masked texts can be generated with assigned labels that are based on the extracted entities of interest as well as a task for which a target NPL model is to be trained.

At block 104, the process 100 includes training an embedding model to capture semantic richness of biomedical context. As will be described in more detail below with reference to FIG. 2a, training the embedding model can include finetuning an LLM or other machine learning model that was already pretrained at least for general-purpose language understanding. The pretraining may or may not involve using data of biomedical context (e.g., obtained from the same or different EHR system). The finetuning of the LLM or other model adapts the embedding model to the specificities of the clinical domain and the particular task for which the target NLP model is to be trained. Illustratively, finetuning the LLM or other machine learning model in this manner (e.g.) enables it to capture a desired semantic space in biomedical context more adequately and effectively than the pretrained general-purpose model.

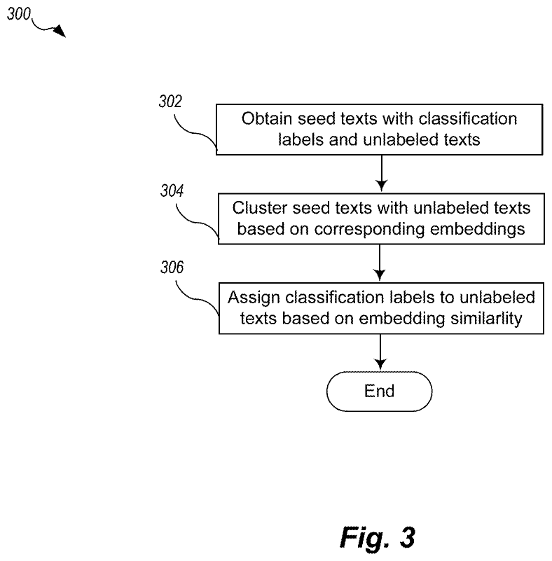

At block 106, the process 100 includes assigning classification labels to unlabeled texts based on embedding similarity. For an unlabeled text (e.g., unstructured text obtained from the EHR system), an embedding representation (e.g., a vectorized semantic representation) can be generated by inputting the unlabeled text into the embedding model. Classification labels can be assigned to unlabeled texts based on their similarity to labeled seed texts in the space of embedding representations. More details will be described with reference to FIG. 3.

At block 108, the process 100 includes providing NLP model training data including the assigned classification labels and their underlying texts. FIG. 1b shows an example tabular format of the NLP model training data. As illustrated, each datapoint (e.g., each row of data) in the NLP model training dataset can include input text (e.g., a context snippet that was initially unlabeled), its embedding representation (e.g., a vectorized semantic representation), and its assigned classification label. This dataset can be used to train the target NLP model (e.g., a support vector machine (SVM), neural network, LLM, or the like) for the task desired.

As an example, training of the NLP model can be based on:

Input: the input text (e.g., a context snippet from the clinical note) and its embedding (e.g., a vectorized representation of the input text), and

Output: the corresponding assigned classification label (e.g., a binary label indicating the result).

The assigned classification labels can be the same as the labels for the desired classification task of the target NLP model. Illustratively, if the desired task of the target NLP model is cancer diagnosis, classification label “positive” or “1” can indicate that the mentioned cancer type in the input text is the patient's diagnosis, while classification label “negative” or “0” can indicate that the mentioned cancer type is not related to the patient's diagnosis. Illustratively, if the desired task of the target NLP model is biomarker detection in input text, classification labels can indicate whether the patient's test result is positive or not positive.

Thus, in some embodiments, this process for enhancing NLP model training can automatically populate large-scale datasets for training at least classification models on medical or clinical notes. In some embodiments, at least a subset of the assigned classification labels is used to generate, modify, or supplement structured texts in the EHR system. In some embodiments, at least a subset of the assigned classification labels is used in conjunction with data obtained from EHR system as input into at least one of a classification, prediction, or association model to produce output.

FIG. 2a is a flow diagram illustrating a process 200 for training and using an embedding model to capture semantic richness (e.g., a desired semantic space) of biomedical context according to some embodiments of the present disclosure. The process 200 may be performed using one or more real or virtual machines and/or one or more containerized applications, inside or outside of the EHR system.

The process 200 starts at block 202, where entities of interest are identified or otherwise extracted from unstructured texts. As described above, the unstructured texts can include context snippets derived from medical or clinical notes that are obtained from the EHR system. In some embodiments, each contextual snippet can include a number of tokens, starting from the left side of a center entity of interest, including the center entity itself, and extending a specified number of tokens (e.g., 40 tokens) to the right side of the center entity. A token can refer to a single, atomic unit of a sequence of characters in the text. It can represent individual words, punctuation marks, or even subwords if the text has been processed at a more granular level. Tokens are typically the building blocks used to break down a sentence or a document into smaller parts for analysis, e.g., via a tokenization process.

In some embodiments, the entities of interest are identified or extracted in accordance with a dictionary, ontology, or other collection of terminologies in biomedical context. In some embodiments, the entities of interest for extraction are selected based on a task of the target NLP model to be trained. The task for the NLP model includes predicting patient information including their demographics, medical history, family medical history, diagnoses, medications, treatments, therapies, procedures, and/or clinical trials; estimating responses to medications, treatments, therapies, procedures, and/or clinical trials, identifying biomarkers, recommending medications, treatments, therapies, procedures, and/or clinical trials, combination of the same or the like. The identifying or extracting of the entities of interest can be based on regular expressions, NLP, or other modeling methods.

As used herein, a biomarker can refer to one or more biological molecules associated with a particular disease or condition, and/or indicative of a particular cell type, cell state, tissue type, or tissue state. Biomarkers can include, for example, nucleic acids, proteins, lipids, sugar moieties, hormones, and the like. Biomarkers can be used as part of a predictive, prognostic, or diagnostic process. For example, biomarkers may be used to predict the likelihood that a particular patient or subject will respond to a particular therapeutic. In some cases, the mere presence (or absence) of a biomarker in a biological sample is indicative of a particular condition, whereas in other cases the biomarker is only indicative of a condition when it is present at a particular level or in a specific location within a biological sample. For example, in some cases a biomarker is a differentially expressed gene. In some embodiments, the biomarker is a therapeutic target. In some embodiments, the biomarker is a cancer biomarker, that is, a biomarker that is associated with cancer.

At block 204, the process 200 includes replacing the extracted entities of interest with masks to generate masked texts. Based on the intended task of the target NLP model, specific entities of interest can be selected and replaced with one or more types of mask tokens. For example, if the task is about identifying cancer diagnosis, entities pertaining to cancer types would be masked. If the task is about extracting genetic test results, then entities related to biomarkers would be masked. In some cases where the task involves associations between or among multiple concepts, multiple types of mask tokens can be used.

At block 206, the process 200 includes assigning labels to masked texts. The labeling can be based on the task of the target NLP model and the masked entities of interest. In various embodiments, the extracted and now-masked entities of interest can be normalized into concepts based on medical or clinical ontology (e.g., Unified Medical Language System (UMLS)) or other hierarchy of biomedical terminology. For example, all mentions/lexical variants “NSCLC,” “Non-small cell lung cancer,” or “Non-Small Cell Carcinoma of the Lung” can be normalized to the concept “Non-Small-Cell Lung Carcinoma.” Each masked text can be labeled by the normalized concept of the masked entity of the text.

FIG. 2b shows two examples of masked texts and their assigned labels. The first example is based on a diagnosis task for the target NPL model, and the second example is based on a biomarker detection task for the target NPL model. As shown, each masked text in the “masked_context” column includes an exemplary mask token “[MASK]” that has replaced an extracted entity of interest, and the masked text's assigned label is indicated in the “concept” column. In some embodiments, a same unstructured text can correspond to multiple instances of masked texts each associated with different masking or labeling.

Referring back to FIG. 2a, at block 208, the process 200 includes finetuning a pretrained LLM (e.g., a BERT or GPT model) to predict the assigned labels in the masked texts. As described above, the LLM was pretrained at least for general-purpose language understanding. The pretraining may or may not involve using data of biomedical context (e.g., obtained from the same or different EHR system). The finetuning of the LLM adapts the embedding model to the specificities of the clinical domain and the particular task for which the target NLP model is to be trained. The finetuning can include adapting the LLM to use each masked text to predict its associated target label, and the parameters of the LLM are adjusted during the adapting and fixed after the adapting is completed (e.g., based on a satisfactory cross-validation result).

At block 210, the process 200 includes using the finetuned LLM to generate embedding representations. Illustratively, given an input (e.g., masked text) to the finetuned LLM, a vectorized semantical representation of the input can be generated based on output of one or more layers of the finetuned LLM, to represent semantic meaning of the input in biomedical context. In some embodiments, the vectorized semantical representation of the input may be generated from the outputs of any selected layer or combining outputs from two or more layers such as by averaging the outputs of the selected layers (e.g., the first and last layers). FIG. 2c shows an example of an embedding representation (in the “embedding” column) generated for an input text (in the “masked_context” column) as it relates to a corresponding label (in the “concept” column).

FIG. 3 is a flow diagram illustrating a process 300 for assigning classification labels to unlabeled texts according to some embodiments of the present disclosure. The process 300 is used to generate labels for the desired task (e.g., classification) of the target NLP model, based on the embedding trained in using the process 200, with no or minimum manual efforts. The process 300 may be performed using one or more real or virtual machines and/or one or more containerized applications, inside or outside of the EHR system.

The process 300 starts at block 302, where a seed set of seed texts with classification labels as well as an unlabeled set of unlabeled texts are obtained. A collection of unstructured texts (e.g., context snippets without classification labels) having the same or similar entities of interest as used in the training of the embedding model are obtained, e.g., from the EHR system or other sources. The size of the data obtained can be based on the need for the desired task of the target NLP model. For each unstructured text, its corresponding entity (or entities) of interest is masked to create a masked text, and the trained embedding model is applied to generate the embedding (e.g., a vectorized semantical representation) for the unstructured text.

The seed set can be derived from the collection of unstructured texts. The seed texts can be positive or negative exemplars or representatives of the kind of information the target NPL model is intended to identify or categorize. The seed texts can be identified by using some simple but highly confident rules, such as regular expression patterns derived based on knowledge. For example, if the desired task for the target NPL model is to identify cancer diagnosis, then a “negative” or “0” label will be assigned as the classification label for a seed text because it is describing a published study rather than a patient's clinical information containing potential diagnosis. In some embodiments, various manual or semi-manual labeling of the seed texts can be performed in order to obtain the initial seed set, where each seed text can have a highly confident classification label, but the seed text may be less generalized. The unstructured texts that remain unlabeled can constitute the initial unlabeled set.

At block 304, the process 300 includes clustering seed texts with unlabeled texts based on their corresponding embeddings. Iterative clustering can be performed while expanding the seed set. For example, by computing distance or other similarity measures between the embeddings, a list of unlabeled texts can be clustered around the initial seed set. These clusters can be based on the presence of both positive and negative seeds, only positive seeds, only negative seeds, or neither. A subset of unlabeled texts in the cluster(s) that does not contain any seeds, positive or negative, can be sampled and each assigned a label (e.g., positive or negative) based on automatic or manual review, and the reviewed texts can be added into the seed set. After that, another round of clustering can be performed based on the expanded seed set. Multiple rounds of such clustering and seed set expansion can be performed until the seed set is finalized. Finalization of the seed set can be based on one or more criteria. For example, the final seed set is achieved when each and every cluster contains at least one seed.

Performing review on cluster(s) without any seeds to expand the seed set can be based on: 1) ambiguity resolution-clusters that do not align closely with existing positive or negative seeds might represent ambiguous or borderline cases, so reviewing these clusters can provide clarity and ensure correct label assignments, and/or 2) discovering new information—these clusters might contain novel patterns or information not previously covered by the initial seeds or seeds in the previous round, so reviewing them can help identify new seeds or insights that the original/prior seeding process missed.

At block 306, the process 300 includes assigning classification labels to unlabeled texts based on embedding distance or similarity to seeds. In some embodiments, the label assigning can be conducted based on Approximate Nearest Neighbor (ANN) searches. For each unlabeled text (e.g., that remains in the unlabeled set), K closest seeds in the final seed set can be identified based on distances or similarities computed using the embedding of the unlabeled text and the embeddings of the seeds. The classification label for the unlabeled text can be determined based on the labels of the K nearest seeds (e.g., based on a majority of them). Various K values (e.g., 1, 5, 9) can be used to optimize the labeling accuracy.

In some embodiments, the K value is determined by treating at least a subset of the seeds as unlabeled texts, and comparing the ANN-based classification labels with the actual seed labels to achieve maximum accuracy. Illustratively, for each unlabeled text, the assigned label can be determined through a majority vote among the K nearest seeds. If the majority of votes are negative labels, the text is labeled as negative, and vice versa. To determine an optimal K value, the majority vote strategy can be applied to each seed, obtaining a label based on the remaining seeds. This process allows for calculation of Precision, Recall, and F1 score for each K, using the labels obtained from the votes of the nearest K seeds and the actual labels from seeds (e.g., in the initial seed set). The optimal K value can selected based on the F1 scores.

Generally speaking, after the process 200 trains the embedding model, the process 300 involves collecting initial examples (seeds), refining them through iterative clustering with embeddings, and then leveraging the ANN method (or other embedding proximity method) to weakly label data points based on their similarity to the established seeds.

In some embodiments, as an alternative to training/finetuning of an embedding model, topic modeling techniques can be used. In these embodiments, the following process can be implemented:
Step 1: Text Preprocessing with tokenization and stop word removing.
Step 2: Topic Modeling:
1) Latent Dirichlet Allocation (LDA): Apply LDA, a probabilistic topic modeling technique, to extract underlying topics from the text snippets. This helps identify the latent topics within the snippets; or
2) Non-Negative Matrix Factorization (NMF): Alternatively, use NMF, which factorizes the snippet matrix into topic and document matrices, revealing latent topics.
Step 3: Topic Representation based on Topic-Document Distribution: Obtain the topic distribution for each snippet, indicating the presence and strength of various topics within each snippet.
Step 4: Similarity Measurement and clustering, similar to the process 300 described above

The main difference between using topic modeling techniques and using fine-tuned embeddings can be the representation of text. The topic modeling approach can use probabilistic representation, which aims at identifying latent topics or themes within text collections, providing a higher-level, probabilistic view of the content's main ideas, allowing for interpretability and thematic clustering. While fine-tuned embedding can use semantic representation, which can be finetuned for a specific desired task, enabling transfer learning and adaptation to various downstream NLP tasks.

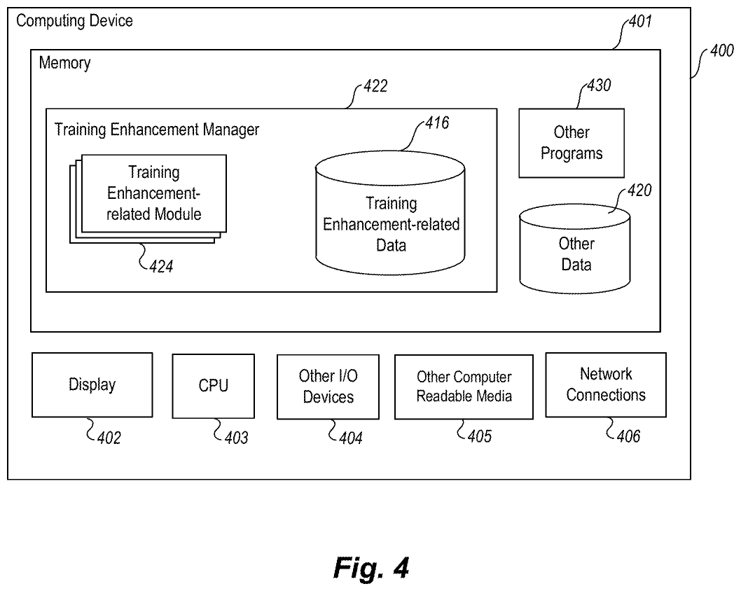

FIG. 4 is a block diagram illustrating a computing system or device 400 used to implement some or all the functionalities of the technology disclosed herein. According to some embodiments, one or more general purpose or special purpose computing systems or devices may be used to implement the computing device 400. In addition, according to some embodiments, the computing device 400 may comprise one or more distinct computing systems or devices and may span distributed locations. Furthermore, each block shown in FIG. 4 may represent one or more such blocks as appropriate to a specific embodiment or may be combined with other blocks. Also, the training enhancement manager 422 may be implemented in software, hardware, firmware, or in some combination to achieve the capabilities described herein.

As shown, the computing device 400 includes a non-transitory computer memory (“memory”) 401, a display 402 (including, but not limited to a light emitting diode (LED) panel, cathode ray tube (CRT) display, liquid crystal display (LCD), touch screen display, projector, etc.), one or more Central Processing Units (“CPU”) or other processors 403, Input/Output (“I/O”) devices 404 (e.g., keyboard, mouse, RF or infrared receiver, universal serial bus (USB) ports, High-Definition Multimedia Interface (HDMI) ports, other communication ports, and the like), other computer-readable media 405, and network connections 406. The training enhancement manager 422 is shown residing in memory 401. In other embodiments, some portion of the contents and some, or all, of the components of the training enhancement manager 422 may be stored on or transmitted over the other computer-readable media 405. The components of the computing device 400 and training enhancement manager 422 can execute on one or more CPUs 403 and implement applicable functions described herein. In some embodiments, the training enhancement manager 422 may operate as, be part of, or work in conjunction or cooperation with other software applications stored in memory 401 or on various other computing devices. In some embodiments, the training enhancement manager 422 also facilitates communication with peripheral devices via the I/O devices 404, or with another device or system via the network connections 406.

The one or more training enhancement-related modules 424 are configured to perform actions related, directly or indirectly, to model training enhancement or other functionalities disclosed herein. In some embodiments, the training enhancement-related module(s) 424 stores, retrieves, or otherwise accesses at least some training enhancement-related data on some portion of the training enhancement-related data storage 416 or other data storage internal or external to the computing device 400.

Other code or programs 430 (e.g., further data processing modules, a program guide manager module, a Web server, and the like), and potentially other data repositories, such as data repository 420 for storing other data, may also reside in the memory 401, and can execute on one or more CPUs 403. Of note, one or more of the components in FIG. 4 may or may not be present in any specific embodiment. For example, some embodiments may not provide other computer-readable media 405 or a display 402.

According to some embodiments, the computing device 400 and training enhancement manager 422 include API(s) that provides programmatic access to add, remove, or change one or more functions of the computing device 400. In some embodiments, components/modules of the computing device 400 and training enhancement manager 422 are implemented using standard programming techniques. For example, the training enhancement manager 422 may be implemented as an executable running on the CPU 403, along with one or more static or dynamic libraries. In other embodiments, the computing device 400 and training enhancement manager 422 may be implemented as instructions processed by a virtual machine that executes as one of the other programs 430. In general, a range of programming languages known in the art may be employed for implementing such example embodiments, including representative embodiments of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), or declarative (e.g., SQL, Prolog, and the like).

In a software or firmware embodiment, instructions stored in a memory configure, when executed, one or more processors of the computing device 400 to perform the functions of the training enhancement manager 422. In some embodiments, instructions cause the CPU 403 or some other processor, such as an I/O controller/processor, to perform at least some functions described herein.

The embodiments described above may also use well-known or other synchronous or asynchronous client-server computing techniques. However, the various components may be implemented using more monolithic programming techniques as well, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs or other processors. Some embodiments may execute concurrently and asynchronously, and communicate using message passing techniques. Equivalent synchronous embodiments are also supported by a training enhancement manager 422 embodiment. Also, other functions could be implemented or performed by each component/module, and in different orders, and by different components/modules, yet still achieve the functions of the computing device 400 and training enhancement manager 722.

In addition, programming interfaces to the data stored as part of the computing device 400 and training enhancement manager 422, can be available by standard mechanisms such as through C, C++, C#, and Java APIs; libraries for accessing files, databases, or other data repositories; scripting languages such as XML; or Web servers, FTP servers, NFS file servers, or other types of servers providing access to stored data. The model-related data storage 416 and data repository 420 may be implemented as one or more database systems, file systems, or any other technique for storing such information, or any combination of the above, including embodiments using distributed computing techniques.

Different configurations and locations of programs and data are contemplated for use with techniques described herein. A variety of distributed computing techniques are appropriate for implementing the components of the illustrated embodiments in a distributed manner including but not limited to TCP/IP sockets, RPC, RMI, HTTP, and Web Services (XML-RPC, JAX-RPC, SOAP, and the like). Other variations are possible. Other functionality could also be provided by each component/module, or existing functionality could be distributed amongst the components/modules in different ways, yet still achieve the functions of the training enhancement manager 422.

Furthermore, according to some embodiments, some or all of the components of the computing device 400 and training enhancement manager 422 may be implemented or provided in other manners, such as at least partially in firmware or hardware, including, but not limited to one or more application-specific integrated circuits (“ASICs”), standard integrated circuits, controllers (e.g., by executing appropriate instructions, and including microcontrollers or embedded controllers), field-programmable gate arrays (“FPGAs”), complex programmable logic devices (“CPLDs”), and the like. Some or all of the system components or data structures may also be stored as contents (e.g., as executable or other machine-readable software instructions or structured data) on a computer-readable medium (e.g., as a hard disk; a memory; a computer network, cellular wireless network or other data transmission medium; or a portable media article to be read by an appropriate drive or via an appropriate connection, such as a DVD or flash memory device) so as to enable or configure the computer-readable medium or one or more associated computing systems or devices to execute or otherwise use, or provide the contents to perform, at least some of the described techniques.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method for enhancing natural language processing (NLP) model training in biomedical context, the method comprising:
training an embedding model to capture semantic richness of biomedical context based, at least in part, on a set of unstructured texts obtained from an electronic health record (EHR) system;
obtaining a seed set including seed texts each associated with a classification label;
obtaining an unlabeled set including unlabeled texts;
determining, via the trained embedding model, a vectorized semantic representation for each seed text of the seed set and for each unlabeled text of the unlabeled set;
assigning classification labels to at least a subset of the unlabeled set by clustering the seed set with the unlabeled set based, at least in part, on the vectorized semantic representations for each seed text and for each unlabeled text; and
providing NLP model training data including the assigned classification labels.

2. The method of claim 1, wherein the set of unstructured texts includes context snippets identified from clinical notes.

3. The method of claim 2, wherein the context snippets are identified based, at least in part, on at least one of demographics, medical history, diagnosis, severity of disease, medication, therapy, surgery, or associated outcome.

4. The method of claim 1, wherein training the embedding model comprises using at least the set of unstructured texts to finetune a large language model (LLM), wherein the LLM was pretrained for general-purpose language understanding.

5. The method of claim 4, further comprising, for each unstructured text of the set of unstructured texts:
extracting one or more entities of interest from the unstructured text; and
replacing at least a subset of the one or more extracted entities of interest with one or more types of mask tokens in the unstructured text to generate respective masked text.

6. The method of claim 5, wherein the at least a subset of the one or more extracted entities is selected based, at least in part, on a task of an NLP model to be trained with the NLP model training data.

7. The method of claim 6, wherein the task of the NLP model includes at least one of predicting diagnoses, identifying biomarker, recommending treatment, or determining medication intake.

8. The method of claim 5, further comprising labeling each masked text with at least one target label based, at least in part, on the one or more entities of interest extracted from the unstructured text.

9. The method of claim 8, wherein the labeling comprises normalizing the one or more entities of interest based, at least on part, on medical or clinical ontology.

10. The method of claim 8, further comprising adapting the LLM to use each masked text to predict its associated target label.

11. The method of claim 10, wherein the parameters of the LLM are adjusted during the adapting and fixed after the adapting is completed.

12. The method of claim 1, wherein given an input to the trained embedding model, a vectorized semantic representation of the input is generated based, at least in part, on output of one or more layers of the trained embedding model.

13. The method of claim 12, wherein the vectorized semantic representation of the input is generated by averaging the output of the one or more layers.

14. The method of claim 1, wherein assigning classification labels to at least a subset of the unlabeled set by clustering the seed set with the unlabeled set comprises iteratively performing the clustering while expanding the seed set.

15. The method of claim 1, wherein the classification labels assigned to the at least a subset of the unlabeled set is based, at least in part, on one or more nearest neighbors in a finalized seed set.

16. The method of claim 1, wherein at least a subset of the assigned classification labels is used to generate, modify, or supplement structured texts in the EHR system.

17. The method of claim 1, wherein at least a subset of the assigned classification labels is used in conjunction with data obtained from EHR system as input into at least one of a classification, prediction, or association model to produce output.

18. The method of claim 1, wherein the NLP model training data is used to train at least one of a support vector machine (SVM), neural network, or large language model (LLM).

19. A computing system for enhancing natural language processing (NLP) model training in biomedical context, the computing system comprising:
one or more processors; and
one or more non-transitory computer-readable media collectively storing instructions that, when collectively executed by the one or more processors, cause the computing system to perform actions, the actions comprising:
training an embedding model to capture semantic richness of biomedical context based, at least in part, on a set of unstructured texts obtained from an electronic health record (EHR) system;
obtaining a seed set including seed texts each associated with a classification label;
obtaining an unlabeled set including unlabeled texts;
determining, via the trained embedding model, a vectorized semantic representation for each seed text of the seed set and for each unlabeled text of the unlabeled set; and
assigning classification labels to at least a subset of the unlabeled set by clustering the seed set with the unlabeled set based, at least in part, on the vectorized semantic representations for each seed text and for each unlabeled text.

20. A non-transitory processor-readable storage medium that stores computer instructions that, when executed by one or more processors, cause the one or more processors to perform actions comprising:
training an embedding model to capture semantic richness of biomedical context based, at least in part, on a set of unstructured texts obtained from an electronic health record (EHR) system;
obtaining a seed set including seed texts each associated with a classification label;
obtaining an unlabeled set including unlabeled texts;
determining, via the trained embedding model, a vectorized semantic representation for each seed text of the seed set and for each unlabeled text of the unlabeled set; and
assigning classification labels to at least a subset of the unlabeled set by clustering the seed set with the unlabeled set based, at least in part, on the vectorized semantic representations for each seed text and for each unlabeled text.
